SEQUENCE LISTING (4 sequences)

SEQ ID NO: 1 - DNA, 4163 bp, Homo sapiens
  CDS (95)..(2083)
ttgatttggtatagtgggaacatttgctttggagacagatgaactggattctgatcgtgaccctgctattttctccttgtgtgactttggagccatgagaccccagatcctgctgctcctggccctgctgaccctaggcctggctgcacaacaccaagacaaagtgccctgtaagatggtggacaagaaggtctcgtgccaggttctgggcctgctccaggtcccctcggtgctcccgccagacactgagacccttgatctatctgggaaccagctgcggagtatcctggcctcacccctgggcttctacacggcacttcgtcacctggacctgagcaccaatgagatcagcttcctccagccaggagccttccaggccctgacccacctggagcacctcagcctggctcacaaccggctggcgatggccactgcgctgagtgctggtggcctgggccccctgccacgcgtgacctccctggacctgtctgggaacagcctgtacagcggcctgctggagcggctgctgggggaggcacccagcctgcataccctctcactggcggagaacagtctgactcgcctcacccgccacaccttccgggacatgcctgcgctggagcagcttgacctgcatagcaacgtgctgatggacatcgaggatggcgccttcgagggcctgccccgcctgacccatctcaacctctccaggaattccctcacctgcatctccgacttcagcctccagcagctgcgggtgctagacctgagctgcaacagcatcgaggcctttcagacggcctcccagccccaggctgagttccagctcacctggcttgacctgcgggagaacaaactgctccatttccccgacctggccgcgctcccgagactcatctacctgaacttgtccaacaacctcatccggctccccacagggccaccccaggacagcaagggcatccacgcaccttccgagggctggtcagccctgcccctctcagcccccagcgggaatgccagcggccgccccctttcccagctcttgaatctggatttgagctacaatgagattgagctcatccccgacagctttcttgagcacctgacctccctgtgcttcctgaacctcagcagaaactgcttgcggacctttgaggcccggcgcttaggctccctgccctgcctgatgctccttgacttaagccacaatgccctggagacactggaactgggcgccagagccctggggtctctgcggacgctgctcctacagggcaatgccctgcgggacctgcccccatacacctttgccaatctggccagcctgcagcggctcaacctgcaggggaaccgagtcagcccctgtggggggccagatgagcctggcccctccggctgtgtggccttctccggcatcacctccctccgcagcctgagcctggtggataatgagatagagctgctcagggcaggggccttcctccacaccccactgactgagctggacctttcttccaatcctgggctggaggtggccacgggggccttgggaggcctggaggcctccttggaggtcctggcactgcagggcaacgggctgatggtcctgcaggtggacctgccctgcttcatctgcctcaagcggctcaatcttgccgagaaccgcctgagccaccttcccgcctggacacaggctgtgtcactggaggtgctggacctgcgaaacaacagcttcagcctcctgccaggcagtgccatgggtggcctggagaccagcctccggcgcctctacctgcaggggaatccactcagctgctgcggcaatggctggctggcagcccagctgcaccagggccgtgtggacgtggacgccacccaggacctgatctgccgcttcagctcccaggaggaggtgtccctgagccacgtgcgtcccgaggactgtgagaaggggggactgaagaacatcaacctcatcatcatcctcaccttcatactggtctctgccatcctcctcaccacgctggccgcctgctgctgcgtccgccggcagaagtttaaccaacagtataaagcctaaagaagccgggagacactctaggtcagtgggggagcctgaggtacagagaagagtgaggactgactcaaggtcacacagtgatccggatcccagaactctggtctccaaattacagcccaggacacctttctctgccgcctgctgcatcagtgggtgacccccttcccgggctgcactttgggtccagctgtggaagccagaagttgggcggtttcagggacagccgagaataatgttgacctgtcagatcaacaaatcttcactgagcatgtattttgtgccacaccctgctctgggcactgggaatgctgggaaatgagatacattcccgccctcaagaatctcccagtctggtaggagagagtgctgcagagccacgtggccgccacgcagtgtgcttagggcctgaggtgtgaaagcccagggctccagagctcggcaggccccgctggtttggtgcggtgagtcctgccccggctgtgcagggtgagggagggccaagccaggaggatttgtctgagacatttccaagcagactgtttgtcacgtcttctgagaatgactttcagtctctctgaaaatgaaaagcttaggaccggaagagagaattggagctgtacgagtgtgtctcggatctggtattgttaggtgggccacggcggctccagcagggtctggttaaggggtccagcccagcactggaccattccgtctcctgctctggacttgccctctcccttcctggcactctcatgttgcataccctgaccccagtgctgctctaagcaccgtccctgcccagccccacttctccatcgcagccccaccttggctgctgagccaggagctaaaaccttagatatctggttctgttttgcacccagcttggcagatgtggatttgaatccaagccttgtgtctgcccctatgtgacagctctatattttatccccgttttataaaagaggaaactgaagttctgaaaatctccttccagggccccagctaactaatgccataggtgagattcaaaccttcatccttctgtctccagggcctgatctttaccactgcaggggctgcaggccgttaagtggacaggaagtggccccacatagcccgagcagggtctggaagcatcctgtgctgtgcacacctgctctctcctctctcccaggcaggcagctgcaggcgctctcctccttctctgcctgtttccctcctcccttcctttccaccctggtgtgggttctcctgttctctctgtgctcttgcattctctcattcccttttcctctatggagcagagcctggagtttgagactatggaatccaacctccccattgcacagatggggaaactgaggcttaggaagagaatgaaacttgtggagagcttatacagaacctctgggggaaaaaagagcccttatttgtggggtgagattgggggttggaccagagtgatgtcctctctcagctatcacatcacaagataatgctggctccaaacttcctttctgtgcctcatcatgcaaggatcttttttccctcttacaaaaacaggtaaaaagcctcacccagatgacccccatccctcataccatggagtcatgagctgtctgggaagaatggacgtgctgggaccaactcaagaccttgttttgctgtcttcatcatcttacctgtgcttggcccacagtctggctcatgatgtgggctcagtaatgtgcgagaaagtgaaaatgccactctctccaccccattttacagaggagaacaccaaggcccagaggaagttaagggagagtcaatgggcagagccagggctaggccctggtggtgtgtggagcacccaggcagacccagtcctggttgggatcacacccacgggtgctactgcacgtaacactcctccttaggcctggaggccaaggtgtgggtccccacgcctgatctttgaaaacactacacagggctgctgtcacttcccagggcccaggcctcagcccaggcctcgggaccaactctttgtataacctacctgaatgtattaaaaactaattttggaaaaaaaaaa

SEQ ID NO: 2 - protein, 662 aa, Homo sapiens
MRPQILLLLALLTLGLAAQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 3 - DNA, 20 bp, Artificial Sequence
  GARP forward primer
gtgactttggagccatgaga

SEQ ID NO: 4 - DNA, 24 bp, Artificial Sequence
  GARP backwards primer
tctcccggcttctttaggctttat

(19) United States
(12) Patent Application Publication
(10) Pub. No.: US 2011/0086367 A1
(43) Pub. Date: Apr. 14, 2011

(54) PHARMACEUTICALS FOR INFLUENCING THE REACTION OF THE HUMAN IMMUNE SYSTEM

(75) Inventors: Michael Probst-Kepper, Braunschweig (DE); Frank Ocklenburg, Tubingen (DE); Darius Moharregh-Khiabani, Hannover (DE); Robert Geffers, Hohenhameln (DE)

(73) Assignees: MEDIZINISCHE HOCHSCHULE HANNOVER, Hannover (DE); HELMHOLTZ-ZENTRUM FUR INFEKTIONSFORSCHUNG GMBH, Braunschweig (DE)

(21) Appl. No.: 12/294,987

(22) PCT Filed: Apr. 3, 2007
(86) PCT No.: PCT/EP07/53246

§ 371 Date: Nov. 21, 2008

(30) Foreign Application Priority Data
Apr. 3, 2006 (DE) 06112182.8

(51) Int. Cl. C07K 14/47; C12N 5/0783; C12N 15/86; C12Q 1/04; G01N 33/566

(52) U.S. Cl. 435/7.24; 530/395; 435/375; 435/455; 435/34; 435/325

(57) ABSTRACT

The present invention uses the potency and efficacy of human glycoprotein-A repetitions predominant protein (GARP), the gene of which is located on chromosome 11q13-11q14 in the reprogramming of antigen-specific effector T-helper cells, which are CD4⁺, towards a regulatory pheno type of pre-determined suppressor activity. In contrast to the known regulatory protein Foxp3 that only induces an incomplete regulatory phenotype without suppressor function, GARP is more efficient in inducing suppressor activity. Further, the use of GARP in the manufacture of pharmaceutical compositions is provided, allowing the production of antigen-specific T_{reg}-cells, having a predetermined suppressor activity for a specific antigen.

The present invention relates to pharmaceuticals for influencing the reaction of the human immune system towards antigen, especially to pharmaceuticals for the treatment of autoimmune diseases, tumors or immunological rejection of transplants.

Further, the present invention relates to processes for manufacturing those pharmaceuticals for medical use in the treatment of autoimmune diseases, tumors or transplant rejections.

### STATE OF THE ART

It is known that immunological tolerance which is based on the discrimination between self-antigen and non-self antigen is a key feature of the immune system, allowing to identify an antigen (Ag) as non-self for subsequent adverse immune reaction while not attacking an immune reaction against self Ag. Clinical symptoms of a misled immune response for example occur in the development of tumors, wherein a tumor evades an adverse immunological reaction by displaying self-antigen, in autoimmune diseases, which are caused by the immune system falsely recognising self antigen as foreign, and in the rejection of transplants, wherein the natural recognition of non-self antigen, e.g. a foreign HLA (human leukocyte antigen), elicits a strong immune response against the transplant. In transplant rejection, the otherwise healthy and natural immunological rejection of the Host versus Graft (HvG) disease, or in the case of transplantation of cells of the immune system, in the Graft versus Host (GvH) disease, are therapeutically undesired and a pharmaceutical composition for suppressing the GvH and/or HvG disease, respectively, is desirable.

One aspect of the regulation of the immune response is caused by peripheral lymphocytes, which are required to prevent auto-immunoreactivity by T-cells that escaped thymic selection or originated de novo against self-antigen. The regulation of immunological responses is at least in part controlled by regulatory T-cells (T_{reg}), which have been identified to be CD4⁺ and CD25⁺. The regulatory functions of fully functional T_{reg}-cells is to suppress the immune reaction against antigen in other lymphatic cells. In the healthy immune system, this suppressor function of T_{reg}-cells suppresses an immune reaction against self-antigen, e.g. by CD8⁺ and CD4⁺ T-cells, but also against environmental antigen, alloantigen or hapten.

As described by Fontenot et al., Immunity 329-341 (2005), it is generally accepted in the art that Foxp3 is the master regulating protein in CD4⁺ CD25⁺ T_{reg}-cells. Foxp3⁺ T_{reg}-cells are considered essential, e.g. a deficiency of Foxp3 is lethal in the development of mice and leads to a severe genetic immune defect in humans. Foxp3 controls homoeostasis in the immune system, i.e. the regulation of recognition of non-self and self antigen. Accordingly, T_{reg}-cells have previously also been termed suppressor cells and identified to be CD4⁺ and CD25⁺, with the characteristic marker Foxp3⁺ having been identified only in 2003.

It is known that the human counterpart to naturally occurring CD4⁺ CD25⁺ T_{reg} in mouse, CD4⁺ CD25^{high} T_{reg}, are a fraction of approximately 2-4% of all CD4⁺ T-cells, as CD25 is found on non-regulatory T-cells, but at lower levels than on T-cells having suppressor activity. Accordingly, CD25 is not regarded as a distinct marker for discriminating regulatory from activated non-regulatory T-cells, because CD25 represents an activation marker of T-cells in general (reviewed in Baecher-Allan et al., Curr. Op. in Immun., 214-219 (2006)).

Further it is known that all CD4⁺ CD25^{high} T-cells can also express Foxp3, which is a winged-helix/forkhead transcription factor, but as for CD25 at differing levels, depending on their activation or resting state (cited in Baecher-Allan et al. (2006)) and depending on their origin of being CD4⁺ CD25^{high} T_{reg} cells or activated non-regulatory CD4⁺ CD25⁺ T-cells. For a review, see Ziegler et al., Ann. Rev. lmmunol. 209-226 (2006). In humans, CD4⁺ CD25⁺ Foxp3⁺ T_{reg} have been identified to originate in the thymus, but peripheral origin is also discussed in the art. The expression of Foxp3 is known to be constant and constitutive in CD4⁺ CD25^{high} T_{reg}-cells, essentially keeping this regulator of the specific suppressor activity active independent from outer influence.

Fantini et al. (The Journal of Immunology, 5149-5453 (2004)) describe a central regulatory mechanism of the immune response in human CD4⁺ T-cells by TGF13. Fantini et al. show that TGFP induces Foxp3 expression in CD4⁺ CD25⁻ T-cells when concurrently stimulated (αCD3/28 monoclonal antibodies (mAb)) and induces their regulatory properties.

Further, an experimental medical treatment is currently known, seeking to change the regulation of the immune response by T_{reg}-cells by stimulating autologous T_{reg}-cells to specifically suppress the transplant rejection by the host's immune system after their re-administration to the host. For this treatment, T_{reg}-cells are specifically selected according to their display of CD4⁺ and CD25⁺ markers.

WO2006/103639 A2, which was published after the priority date of the present invention, describes that antibodies can be raised against GARP protein, the antibody serving as a GARP-specific affinity ligand for isolating T_{reg}-cells from peripheral blood mononuclear cells (PBMC). T_{reg}-cells are described to be useful for administration to an individual for increasing suppressor activity in the individual. Identification or generation of antigen-specificity in isolated T_{reg}-cells is not described.

In contrast to the hitherto generally accepted superior regulator function of Foxp3, currently regarded as the master regulator protein for the determination of suppressor activity in T_{reg}-cells, the present inventors demonstrate that Foxp3 is itself expressed in dependency from the presence of a regulator protein, which function has not been described previously for that protein.

### OBJECTS OF THE INVENTION

The present invention aims to provide pharmaceutical compositions suitable for the treatment of symptoms having an immunological origin, e.g. autoimmune diseases, tumor development and the rejection of transplants, e.g. both the GvH disease and the HvG disease.

A further object of the present invention is to provide agents and pharmaceutical compositions comprising these agents that can be used for influencing the suppressor activity of T_{reg}-cells, preferably of isolated T_{reg}-cells, or modulate their function in vivo.

Further, it is an object of the present invention to provide the use of a selectable marker which is characteristic for the distinction of unprimed naïve T_{reg}-cells from primed antigen-specific T_{reg}-cells that are already capable of exerting a specific suppressor effect on the immune response. In addition, it is an object of the present invention to provide a method for selecting primed antigen-specific T_{reg}-cells from antigen-specific activated non-regulatory CD4⁺ CD25⁺ T-cells by making use of the absence and presence to the marker, respectively.

It is a further object of the present invention to provide T-cells having regulatory properties as suppressor T-cells by genetic modification.

### GENERAL DESCRIPTION OF THE INVENTION

The present invention provides pharmaceutical compositions and processes for manufacturing these pharmaceutical compositions to achieve the above-mentioned objects.

Further, the present invention provides a method for medical treatment, the treatment comprising the administration of a pharmaceutical composition comprising GARP or of a GARP-encoding nucleic acid construct.

The protein which has been found to induce the constitutive expression of Foxp3 is termed GARP (glycoprotein-A repetitions predominant), the gene of which is located on chromosome 11q13-11q14. The amino acid and DNA sequences of GARP are known under accession number NM_{—}005512 in GenBank for humans, and is also enclosed as Seq. ID No. 2 (amino acid sequence) and Seq. ID No. 1 (DNA sequence). GARP has been found to trigger the constitutive expression of Foxp3 in CD4⁺ T-cells, which are subsequently specifically primed for their suppressor activity to the antigen present. Accordingly, the present invention provides the use of GARP to modulate the function of T-cells towards antigen-specific regulatory activity, especilly of antigen-specific non-regulatory CD4⁺ T-cells or of T_{reg}-cells for medical treatment and for the production of a pharmaceutical composition.

Further, the present invention provides the use of modulator molecules to influence the activity of GARP, e.g. non-biological synthetic molecules or proteinacious and peptidic molecules like e.g. antibodies, preferably monoclonal, and fragments thereof (e.g. F_{ab}). Modulator molecules are selected from agonists, the interaction of which with GARP causes an enhancement of T_{reg}-cell function, and antagonists, the interaction of which with GARP causes a reduction of T_{reg}-cell functions. At present, it is assumed that these agonist or antagonist acitvities are brought about via intracellular signalling of GARP, following interaction with the modulator molecules. These modulator molecules are suitable for specifically modulating the suppressor activity of T-cells because they interact with GARP, which controls the expression of Foxp3 and, as a consequence, the suppressor function of T_{reg}-cells.

Therefore, the present invention provides T-cells having a pre-determined and specifically generated suppressor activity or a specifically reduced suppressor activity, e.g. for use in the manufacture of pharmaceutical compositions for medical use, and the medical treatment using these T_{reg}-cells of the invention.

The present invention uses the potency and efficacy of GARP in the reprogramming of antigen-specific effector T-helper cells, which are CD4⁺, towards a regulatory phenotype of suppressor activity. In contrast to the known regulatory protein Foxp3 that only induces an incomplete regulatory phenotype without suppressor function as e.g. described by Allan et al. (J. Clin. Invest, 2005), GARP is more efficient in inducing suppressor activity. Further, the use of GARP in the manufacture of pharmaceutical compositions allows the production of antigen-specific T-cells, having a predetermined antigen-specific suppressor activity towards a specific antigen.

According to the central regulatory role of GARP, GARP (protein) or nucleic acid encoding GARP can be used to reprogram the function of antigen specific pathogenic T-cells isolated from an individual patient. These pathogenic T-cells are for example self Ag-specific and can be enriched by e.g tetramer technology (tetramer⁺ cells), including Class II tetramer technology, as described by Yang et al. (J. Immunol. 176, 2781-2789 (2006)), Mallone et al. (Blood, 2004-2012 (2005), and Oling et al. (J. of Autoimmunity 25, 235-243 (2005), and including multimer technology.

An alternative method for enriching tetramer+ CD4+ T-cells is described by Day et al. (J. Clin. Invest. 112, 831-842 (2003) “Ex vivo analysis of human memory CD4 T-cells specific for hepatitis virus using MHC class II tetramers”), using magnetic bead technology, e.g. anti-PE beads with mAb and separation using magnetic force (MACS) for cell separation. In the alternative to MACS, fluorescence activated cell sorting (FACS) methods can be employed in conjunction with tetramer technology. When using e.g. these methods, autologous disease-associated pathogenic T-cells manipulated according to the present invention can be re-infused to the patient for generating an antigen specific suppressor activity and, hence, an immune tolerance towards the specific antigen. In this context, disease related Ag for which T-cells having specific suppressor activity according to the invention are generated, are comprised in the group of autoantigens, e.g. identified in diabetes mellitus, or allo-antigen specific T-cells occuring after transplantation causing e.g. the GvH or HvG diseases.

Antigen-specific T-cells can be expanded in vitro antigen-specifically using autologous PBMC in the presence of specific antigen plus IL2, or without antigen-specificity using anti-CD3-antibody covered beads plus anti-CD28-antibody covered beads. Tetramer technology during subsequent cultivation allows to again control antigen specificity and T-cell differentiation, and to isolate desired T-cells, e.g. T_{reg}-cells by FACS or MACS methods, with CD4⁺-tetramer⁺-GARP⁻ identifying effector T-cells.

For reprogramming of antigen-specific T-cells to T_{reg}-cells, transduction with a GARP-encoding nucleic acid contruct can be used to obtain effector CD4⁺ T_{reg}-cells, e.g. retroviral transduction for stimulated T-cells and lentiviral transduction for stimulated or resting T-cells. These methods are advantageous in that no foreign, and therefore potentially immunogenic antigen serving as a selection marker for subsequent cell sorting is necessary, e.g. GFP for marking transduced cells. In the methods according to the invention, GARP itself can be used as a marker for cell sorting using anti-GARP antibody, preferably mAb, for isolating antigen-specific T_{reg}-cells, e.g. by sorting using MACS or FACS.

As an example for an antigen that for the purposes of the invention is considered a pathogenic antigen against which suppressor activity from T-cells according to the invention can be generated, is allogeneic HLA I and HLA II in the case of hematopoietic stem cell transplantation. In this case, GARP is used for the production of a pharmaceutical composition for the treatment of GvH disease using HLA molecules of the transplant recipient as the antigen. Alternatively, in the case of organ transplantation, GARP is used for the production of a pharmaceutical composition for the treatment of HvG disease using HLA molecules of the transplanted organ.

Another exemplary antigen, against which suppressor activity from T-cells according to the invention can be generated, is insulin. Insulin is a preferred antigen for generating suppressor activity as Kent et al., Nature 224-228 (2005) have found that autoimmune type I diabetes is associated with pathogenic T-lymphocytes. T-cells manipulated according to the invention having insulin-specific suppressor activity are useful for producing a pharmaceutical composition for the treatment of autoimmune type I diabetes.

A further exemplary antigen, against which suppressor activity from T-cells according to the invention can be generated, is human autologous myelin basic protein, because at least one aspect of multiple sclerosis is an attack of the immune system against autologous myelin basic protein. Accordingly, treatment with T-cells manipulated according to the invention having autologous myelin basic protein-specific suppressor activity is useful. These T-cells of the invention can be contained in a pharmaceutical composition for the treatment of an autoimmune disease, e.g. type I diabetes.

In this embodiment, the present invention provides a method for manipulating antigen-specific T-cells, e.g. T-helper cells, to generate T-cells having suppressor activity towards the same antigen. These T-cells of the invention have cell contact-dependent suppressor activity, resembling activated CD4⁺ CD25^{high} T_{reg}-cells. In general, this embodiment uses the manipulation of antigen specific non-regulatory T-cells by at least transient contact with or by at least transient expression of GARP within these cells to change them into T-cells having suppressor activity as activated T_{reg}-cells by the induction of Foxp3. Accordingly, the invention provides T-cells having suppressor activity for a predetermined antigen, generated from non-regulatory but Ag-specific T-cells, preferably T-helper cells. According to the pattern of expression products induced by this process, it can also be described as a reprogramming of Ag-specific T-cells to T_{reg}-cells, maintaining their Ag-specificity.

Expression of GARP for manipulating autologous or allogenic naïve unprimed T_{reg}-cells or non-regulatory T-cells can be caused by transient introduction of nucleic acid encoding GARP, e.g. by electroporation (as e.g. described by Fantini et al., J. Immunol. 172, 5149-5153 (2004)) or by retroviral transduction. Expression of GARP, optionally only transient expression, leads to the expression of Foxp3 and drives the differentiation of the respective T-cell species towards suppressor activity. For antigen specific differentiation, naïve unprimed T_{reg}-cells and/or non-regulatory T-helper cells can be contacted with antigen presenting cells of the same or a different donor as previously described by Walker et al. (PNAS 102, 4103-4108 (2005)) in the presence of the antigen against which suppressor activity is desired. When manipulating non-regulatory T-cells, e.g. T-helper cells, Ag-specificity can be present in activated T-cell fractions isolated from patients having an immune related disease, or Ag-specificity can be generated by e.g. contact between professional antigen presenting cells (APC) with non-activated non-regulatory T-cells. T-cells manipulated according to the present invention provide a desired Ag-specific suppressor activity, e.g. by manipulation for at least transient expression of GARP, which directs the effector T-cell differentiation essentially towards the desired antigen-specific regulatory suppressor phenotype.

As it has been found that GARP can be considered a master regulator for Foxp3, with presence of GARP correlating with presence of Foxp3 and correlating with suppressor activity, presence of GARP is preferably constitutive, e.g. by constitutive expression from a GARP-encoding nucleic acid construct. Accordingly, for stable suppressor activity of T_{reg}-cells according to the invention, constitutive expression of GARP is preferred, e.g. genetically manipulated T_{reg}-cells obtainable from retroviral or lentiviral transduction with a nucleic acid construct encoding GARP under the control of a constitutive promoter.

In the methods according to the present invention for specifically separating CD4⁺ GARP⁺ regulatory T-cells, from other e.g. non-regulatory T-cells, for the generation of antigen-specific regulatory CD4⁺ T-cells which may also express the activation marker CD25, the cell-surface molecule GARP serves as specific marker. A single surface-exposed marker for T_{reg}-cells is unknown in the art, as the regulatory protein Foxp3, the high level expression of which is regarded in the art as an essential characteristic for the distinction of regulatory from non-regulatory T-cells (Baecher-Allan et al., loc cit.), is intracellularly located.

In accordance with these findings, due to the absence of GARP on unprimed naïve T_{reg}-cells, these can be separated from T_{reg}-cells which are primed for a specific antigen related suppressor activity and display GARP, as GARP represents an early-induced gene of CD4⁺ CD25^{high}-derived T_{reg}-cells that were antigen-specifically stimulated via their T-cell receptor.

Alternatively, primed T_{reg}-cells having specific suppressor activity can be selected as displaying GARP from patients suffering from immune related disease for selective in vitro expansion of T_{reg}-cells therefrom. One known method for selecting primed from unprimed T_{reg}-cells or separating primed or unprimed T_{reg}-cells from a mixture including activated CD4⁺ CD25⁺ non-regulatory T-cells, is FACS using specific staining to the presence of GARP, e.g. using an anti-GARP antibody coupled with a fluorescence marker. Alternatively, specific antibodies to GARP can be used for separation in an immobilized state, e.g. attached to magnetic beads.

Accordingly, the present invention provides the use of the absence or presence of GARP on T_{reg}-cells for discriminating, e.g. selecting, unprimed naive T_{reg}-cells from primed T_{reg}-cells having suppressor activity, respectively, as well as selecting acitvated non-regulatory CD4⁺ CD25⁺ T-cells according to their expression of GARP, but independent from expressing Foxp3 at different levels.

Further, the present invention provides an antibody having specificity for GARP and the use of an antibody having specificity for GARP in a method for discriminating, e.g. separating naïve unprimed T_{reg}-cells from primed T_{reg}-cells having suppressor activity. Using GARP as the specific marker for primed T_{reg}-cells having suppressor activity from unprimed naïve T_{reg}-cells, which are GARP⁻ and/or the use of an antibody specific for GARP in T_{reg}-cells, a method is provided for discriminating e.g. selecting primed T_{reg}-cells having suppressor activity from unprimed naive T_{reg}-cells.

Here, the present invention offers an advantage over the known state of art as GARP can be used as a specific marker to isolate antigen-specific T_{reg}-cells previously activated by their respecitve antigen, which T-cells could hitherto not easily be distinguished from CD4⁺ CD25⁺ non-regulatory T-cells, which are already primed.

Primed effector CD4⁺ T-cells are potentially harmful contaminants to preparations of T_{reg}-cells exerting a desired antigen specific suppressor activity, which in state of art preparartions could not be identified or eliminated by selection for CD25 overexpression only prior to administration to a patient. Therefore, in medical preparations in the state of art, antigen-specific activated T_{reg}-cells and effector CD4⁺ T-cells can be contained. The latter may counteract the desired suppressor activity from the manipulated T_{reg}-cells, for example after re-administration to an autologous patient (e.g. prevention of HvG), or when introduced into a allogeneic patient (GvH).

The pharmaceutical compositions according to the invention in a first embodiment comprise manipulated T-cells, preferably essentially uncontaminated from T-cells previously activated but lacking suppressor function, i.e. the compositions are essentially free from uncontrolled T-cell effector functions. The compositions comprise regulatory T-cells according to the invention that are specifically activated to suppress an immune reaction against a specified antigen. In this embodiment, the Ag-specificity of the suppressor activity of T-cells according to the invention is based on the induction of suppressor activity within a primed T-cell. The antigen, against which the suppressor activity of T-cells of the invention is generated, can be non-self or self antigen, the latter also termed autologous antigen.

In a second embodiment, the present invention provides pharmaceutical compositions comprising selective regulatory T-cells, their depletion or inactivation for use in the treatment of tumors and vaccination. In cases where existing or arising activation of antigen-specific regulatory T-cells impairs the establishment of an effective immune response, this embodiment provides regulatory T-cells that are selectively targeted by e.g. anti-GARP antibodies that can be coupled to pharmaceuticals, or impaired in their functional activity e.g. by antagonists to GARP or inhibitors to GARP signalling to improve the immunoreaction following anti-tumoral vaccination or vaccination against infectious agents.

In a further embodiment, the present invention provides the selective induction of priming, e.g. activation of the suppressor activity in T-cells, which may be unprimed naive T_{reg}-cells or non-regulatory Ag-primed T-cells, by making use of GARP expression to trigger the constitutive expression of Foxp3, which in turn leads to the induction of Ag-specific suppressor activity, comparable to activated T_{reg}-cells. Expression of GARP in T-cells can for example be achieved by transient transformation using nucleic acid constructs, e.g. DNA or RNA encoding GARP, or viral transfection systems, e.g. retroviral permanent or transient transfection of antigen-specifically activated non-regulatory T-cells for triggering the expression of Foxp3 in CD4⁺ Foxp3⁻-T-cells to reprogram their effector development towards a T_{reg}-cell phenotype.

### DETAILED DESCRIPTION

The present invention is now described by way of examples and in relation to the figures, wherein

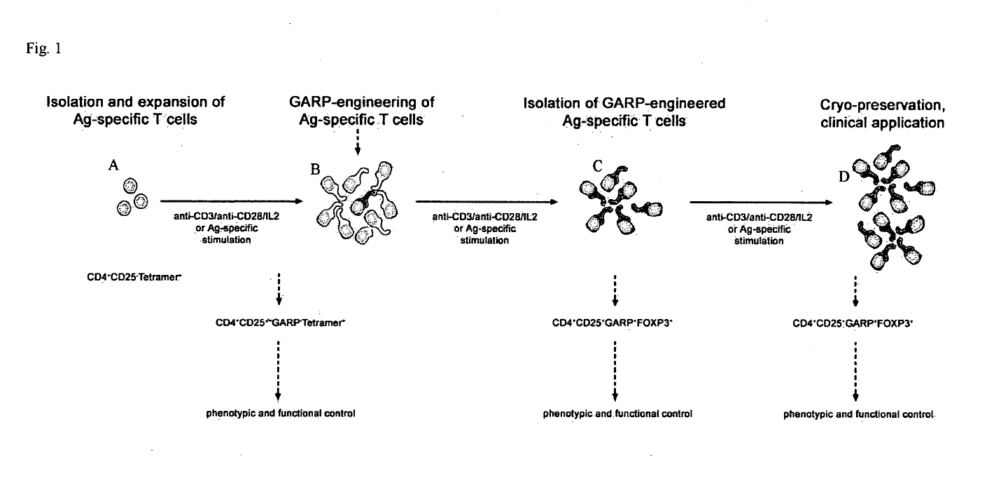

FIG. 1 schematically shows an overview of processes according to the invention for the generation of functional T_{reg}-cells having cell-contact dependent suppressor activity towards a pre-determined antigen,

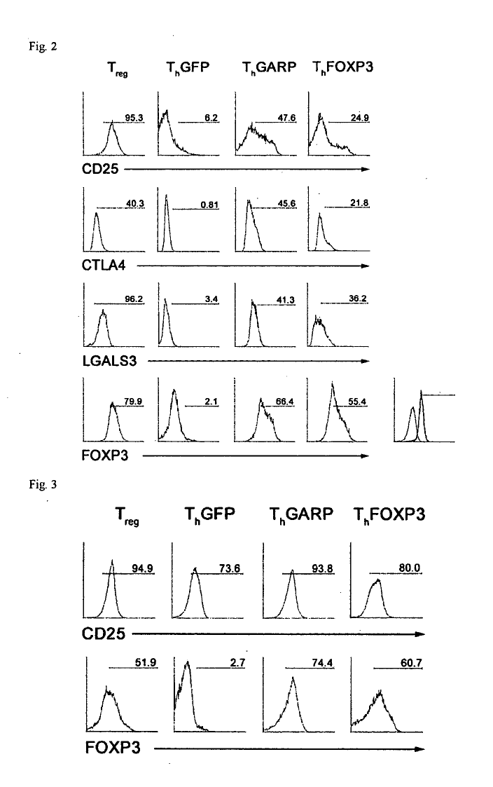

FIG. 2 shows the flow cytometry characterization results of Tₕₑₗₚₑᵣ-cells (Tₕ) transduced with expression cassettes encoding GARP (TₕGARP), Foxp3 (TₕFOXP3) or GFP (TₕGFP), respectively, when using tagged antibodies specific for CD25, CTLA4, LGALS3, and FOXP3, respectively, at day 10 after stimulation,

FIG. 3 shows the analysis of cells used for FIG. 2 after stimulation for 3 days,

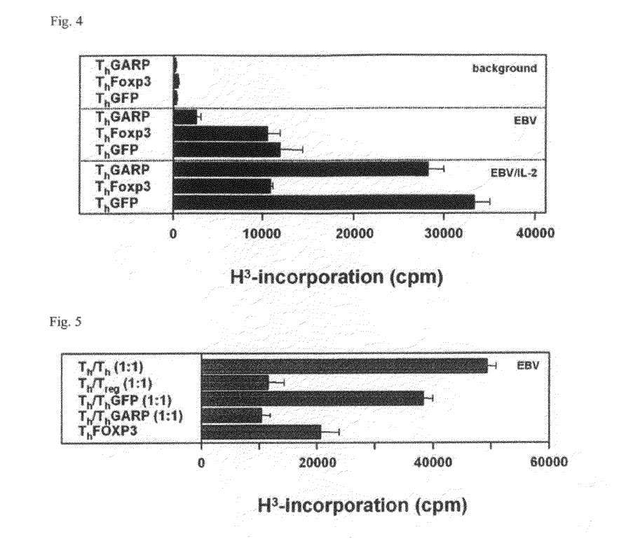

FIG. 4 shows measurement results of ³H-thymidin (cpm) incorporation by T_{reg}-cells obtained by transduction of Tₕ-cells with a GARP encoding expression cassette in comparison to Tₕ-cells after stimulation by irradiated EBV B-cells without and with exogenous IL2 and background at day 3, and

FIG. 5 shows the T_{reg}-cells and Tₕ-cells as in FIG. 4 at day 3 in a test for inhibition of proliferation of Tₕ-cells using alloantigen-stimulation with irradiated EBV B-cells in the presence of irradiated T-cells at a ratio of 1:1. This suppression is cell-contact dependent as it is prevented by a trans-well membrane interrupting cell-contact (data not shown).

As schematically depicted in FIG. 1, functional T_{reg}-cells according to the invention can be generated from antigen-specific Tₕ-cells by expression of GARP, or from Tₕ-cells without antigen-specificity by induction of antigen-specificity using presentation of the antigen against which suppressor activity is desired, by APC, followed by expression of GARP. Preferably following the isolation of CD4⁺ CD25⁻ tetramer⁺ T-cells, preferably in a resting state from a patient, e.g. from PBMC, using tetramer technology as schematically shown in step A (nucleus indicated as circular structure), cells are stimulated in vivo by presence of anti-CD3, anti-CD28, IL2, or antigen-specifically, e.g. by presence of APC provided with a pre-selected antigen. The resultant stimulated T-cells, e.g. Tₕ-cells, are provided with GARP, preferably by viral transduction with a nucleic acid construct comprising an expression cassette encoding GARP. Due to stimulation, cells change their morphology to an enlarged and polymorph shape, as shown from B for all cells. Following contacting with a viral transduction vector, depending on the transduction efficiency only a fraction of the Ag-specific T-cells are effectively transduced to GARP⁺. The resultant effectively transduced GARP⁺ Ag-specific T-cells are shown in B as filled, darker cell. The mixture of non-transduced (light shade cells) and GARP⁺ Ag-specific T-cells can further be expanded by stimulation with anti-CD3, anti-CD28, IL2 and/or specific antigen presented by APC. The effectively transduced GARP⁺ Ag-specific T-cells can be isolated from the admixture with non-transduced cells according to their expression of GARP using an anti-GARP antibody. As shown at C, these isolated cells are CD4⁺ CD25⁺ GARP⁺ Foxp3⁺.

Functional and phenotypic control can be used in all steps to confirm efficiency. These T_{reg}-cells can be used as a pharmaceutical composition for administration to a patient for exerting the Ag-specific suppressor function. For medical application and before preservation, optionally followed by an additional stimulation step with anti-CD3, anti-CD28, IL2 and/or Ag-specific stimulation, Ag-specific T_{reg}-cells can be cryo-preserved and thawed for medical use according to standard cell-culture protocols as indicated in D.

In greater detail, it has been found by the present inventors that human effector CD4⁺ T-cells, e.g. alloantigen-specific CD4⁺ T-helper cells, can be manipulated to have cell contact-dependent suppressor function and T-cell anergy, similar to CD4⁺ CD25^{high} T_{reg}-cells, e.g. after ectopic overexpression of GARP. When over-expressing GARP in human effector T-helper cells, it was found that expression of Foxp3 was up-regulated in combination with an up-regulation of LGMN (cystein endoprotease legumain) and the galectin LGALS3, which both have been identified to be Foxp3 dependent genes expressed at high levels in activated T_{reg}-cells, as well as an up-regulation of UBD, IL1R2 mRNAs, CD25, and CTLA-4. Further, the inhibition of transcription of IL-2 was found following three days of T-cell activation in vitro using anti-CD3 antibodies/and IL-2, consistent with the knowledge that Foxp3 is a repressor of IL-2 transcription. Further, an impairment of T-helper cell proliferation was observed. In summary, the phenotype of human effector T-helper cells was similar to that of activated CD4⁺ CD25^{high} T_{reg}-cells, i.e. having cell contact dependent suppressor function and T-cell anergy, making the use of GARP for at least transient expression, preferably stable expression, in T-cells a valuable tool for general suppressor function in these T-cells in a predetermined way.

From a comparison of the analytical data obtained from the expression of GARP in non-regulatory T-cells to those of CD4⁺ CD25^{high} T_{reg}-cells, it is at present inferred that the manipulation of T-cells according to the invention by the presence of GARP leads to the phenotypically stable suppressor function of both the manipulated T-cells according to the invention and natural T_{reg}-cells. In detail, it is assumed that GARP ensures a stable regulatory phenotype in human effector T-cells, e.g. in T-helper cells, via up-regulation and maintenance of high levels of Foxp3 expression in resting and activated T-cells. This regulation seems to create a feed-forward loop between Foxp3 and GARP, probably assisted by LGALS3 and LGMN. The basis for this assumption is that Foxp3 ensures early up-regulation of GARP in T_{reg}-cells as well as in T-helper cells genetically manipulated to express high levels of Foxp3, both cell types expressing high levels of LGALS3 and LGMN. Over-expression of LGALS3 or LGMN up-regulates transcription of GARP and Foxp3, assisting in high level expression of Foxp3 following T-cells activation. As a consequence, up-regulated gene expression of Foxp3 is ensured by a positive feed-forward circuit of sustained levels of LGMN and LGALS3 expression, both contributing to a sustained expression of GARP and, as a consequence, high protein levels of Foxp3.

When using T-helper cells having an antigen specificity for the generation of effector T-cells having suppressor activity according to the invention, the antigen specificity is maintained.

The present invention is now described in greater detail by way of examples. In the following assays, it could be shown that the suppressor activity present in T-cells, which in the case of human T-helper cells originally were non-regulatory T-cells, manipulated according to the invention maintained their antigen-specificity, whereas in the case of naïve unprimed T_{reg}-cells, antigen specificity could be generated by co-cultivation with APC, presenting the respective antigen.

### EXAMPLE 1

### Isolation of Unprimed Naïve T_{reg}-Cells from Peripheral Lymphocytes

In general, unprimed T_{reg}-cells were isolated from peripheral lymphocytes of a healthy blood donor by FACS, using fluorescence labelled antibodies against CD4 and CD25.

In detail, CD4⁺ T-cells were isolated by centrifugation over Ficoll-Hypaque gradients (Biochrom AG, Berlin, Germany) and enriched using the CD4⁺ MACS isolation kit that depletes most of the non-CD4⁺ T-cells of peripheral blood, e.g. CD8⁺ T-cells, macrophages and dendritic cells, granulocytes and NK cells, and AutoMACS technology (Miltenyi Biotech, Bergisch Gladbach, Germany), followed by separation into fractions of CD4⁺ CD25^{high} and CD4⁺ CD25⁻ T-cells, respectively by FACS (MoFlo, DakoCytomation, Ft Collins, USA) to a purity of >98%. For sorting, cells were stained with anti-CD4-Cychrome and anti-CD25-PE. Following antigen-specific stimulation using professional Ag presenting cells (APC) and the specific Ag for which suppressor activity is desired, T_{reg}-cells were Ag-specifically primed and activated by the respective antigen, up-regulating expression of GARP on the cell surface. These Ag-specifically stimulated GARP⁺ CD4⁺ T_{reg}-cells can be separated from the other cells according to the expression of GARP using an anti-GARP antibody.

The antibody preparation was raised by immunizing a rabbit or mice with GARP or, alternatively, with extracellular regions of GARP, e.g. encoded by aminoacids No. 1-612 of the GARP protein fused to a His-tag for purification in a pcDNA3-derived plasmid and expressed in cell culture in 293 cells or, alternatively, in bacterial expression plasmids, e.g. pET22, for expression in E. coli, strain BL21 and derivates thereof. Isolation of GARP from cell culture supernatant was achieved by binding to a His-tag specific column (Amersham), washing and subsequent elution of GARP. In the case of bacterial expression, isolation of GARP is generally more efficient and more economic.

For FACS, antibody was labelled with FITC and biotin according to standard protocols. Lymphocytes were separated by FACS using a MoFlo or FACS Vantage or ARIA cell sorter (BD Pharmingen).

### EXAMPLE 2

### Isolation of Primed T_{reg}-Cells from Peripheral Lymphocytes

From infiltrating lymphocytes, e.g. transplant rejections, CD4⁺ GARP⁺ T-cells were isolated by subsequent FACS using fluorescence-labelled anti-CD4 and anti-GARP antibodies, respectively. The isolated CD4⁺ GARP⁺ T-cell fraction was expanded in vitro using standard cell cultivation methods. For cell cultivation, RPMI 1640 medium supplemented with 2 nM L-glutamine, 2.5 mM HEPES (Sigma-Aldrich), 100 U/μg/mL penicillin/streptomycin (BioWhittaker), 0.5 mM Na-pyruvate, 0.05 mM non-essential amino acids (Gibco) and 5% human AB serum (Gemini Bio-Products) was used.

As an alternative cultivation protocol, isolated CD4⁺ GARP⁺ T-cells were cultured in X-vivo 15 medium (Cambrex BioWhittaker) with 15% pooled human AB serum, 2 mM glutamine and 20 mM HEPES, supplemented with 2000 IU/mL human recombinant IL-2 (Chiron). Optionally, anti-CD3/anti-CD28 antibody coated beads (Xcyte Therapeutics) were added in a 1:1 ratio of T-cells:beads. After expansion, medium was changed to remove IL-2, and beads were separated by magnetic attraction (magnetic particle concentrator, Dynal).

### EXAMPLE 3

### Isolation of Primed T-Helper Cells from Peripheral Lymphocytes

Peripheral lymphocytes were isolated from patients with an autoimmune disease or transplant patients having developed GvH or HvG disease.

In the alternative, human auto-antigen-specific T-cells were isolated to generate T_{reg}-cells having suppressor activity for that auto-antigen by expression of GARP, using the cloning method as ascribed by Mannering (Journal of Immunological Methods 83-92 (2005)).

T_{reg}-cells having suppressor activity for a specific human auto-antigen can be used for producing a pharmaceutical composition for the treatment of immune diseases. The method for isolating and cloning of Mannering et al. to produce human antigen-specific non-regulator T-cells, which in one embodiment provide the basis for the T_{reg}-cells according to the present invention, could be obtained from PBMC isolated over a Ficoll-Hypaque gradient. After washing the PBMC pool in PBS (phosphate buffered saline), cells were cultured in Iscove's modified Dulbecco's medium (Gibco, Rockville, USA), supplemented with 5% pooled male human serum, 2 mM glutamine (Gibco), 5×10⁻⁵ M 2-mercapto ethanol (Sigma Aldrich), penicillin (100 U/mL), streptomycin (100 μg/mL) and 100 μM non-essential amino acids (Gibco) as a complete culture medium. PBMC were incubated at 1×10⁷/mL in PBS at 37° C. for 5 minutes with 0.5 μM CFSE (Molecular Probes, Eugene, USA) Staining was terminated by adding culture medium containing 5% pooled human serum, washing the cells once in PBS containing 1% pooled human serum and suspending in culture medium at 1.00×10⁶/mL. Stained cells were cultured at 2×10⁵/well in a volume of 115 μL in 96-well round bottom plates (Becton Dickinson, USA) with complete medium, optionally containing the recall antigen tetanus toxin at 10 LFU/mL as a positive control, glutamic acid decarboxylase-65 (GAD) or pro-insulin (10 μg/mL) as model auto-antigens. Unstained cells included in all experiments were used to set compensations of the flow cytometer. After 7 days culture, cells for each antigen were pooled, washed in PBS and stained on ice with anti-human CD4-PE (IgG-2a, clone RPA-T4) (BD Pharmingen, San Diego, USA). Optimal compensation and gain settings were determined for each experiment on the basis of single stained and unstained samples. Sorting was done for single CD4⁺, CFSE-dim-cells (propidium-iodide negative). Each well contained feeder cells, cytokines (10 U/mL IL-2, 5 ng/μL IL-4, 5 ng/mL IL-7, 5 ng/mL IL-15) and mitogen (2.5 μg/mL PHA, 30 ng/mL anti-CD 3, 100 ng/mL anti-CD28). All cultures contained amphothericin B at 2 μg/mL. Cells were fed every seven days with fresh cytokines in 50 μL of medium. After about 2 weeks, clones were expanded into 48 well plates and tested for antigen-specificity by ³H-thymidine incorporation assays. Clones with a stimulation index (CPM with antigen/CPM without antigen (counts per minute)) at or above 3 were expanded with PHA, IL-2, IL-4 and feeder cells as described above, or with anti CD3, using full medium containing IL-2 plus IL-4 instead of only IL-2.

Antigen-specific T-cells were identified by their reduction in CFSE staining during culture with antigen. Flow cytometer gates were set to exclude dead cells and doublets, sorting CD4⁺, CFSEdim-cells singly into wells containing cytokines, mitogen and feeder cells. For confirmation of expressing a single T-cell receptor (TCR) VR gene, PCR amplification of the Vβ gene was used by amplifying a fragment of the Vβ region.

From this pool of lymphocytes, antigen-specific effector cells were identified as e.g. described by Kent et al., Nature 224-228 (2005). In detail, peripheral lymphocytes were isolated over Ficoll-Hypaque gradients or, alternatively obtained from draining lymph nodes or spleen. T-cells were cloned at 0.3 cells/well, with 3 μg/mL phytohaemagglutinin (PHA-P, obtained from Remel) and irradiated allogeneic PBMCs and 20 U/mL recombinant human IL-2 (Tecin, obtained from NCI) in the presence of 10 μg/mL anti-Fas antibody (Boehringer Ingelheim, Germany) to prevent death of reactivated T-cells when activated with allogeneic feeders and PHA. Medium for T-cell cultures contained 5% heat inactivated human male AB serum (Omega scientific) in RPMI 1640 with 10 mM HEPES buffer, 2 mM L-glutamine, 10 U/mL penicillin and 100 μg/mL streptomycin (all Cambrex bioscience). T-cell clones were expanded with IL-2, assayed on day 9 or 10 following stimulation, and re-stimulated as previously described by Hafler et al. (J. Exp. Med. 1625-1644 (1988)).

Antigen reactivity was examined using irradiated (5,000 rads) B-cells pulsed with antigenic peptide (250 μM) for 2 hours, washed and plated in triplicate at approximately 50,000 cells/well with equal numbers of T-cell clones. Each T-cell clone was also applied plated onto plate-bound anti-CD3 antibody (OKT3 at 0.05 μg/well) to assess the viability of each clone in each experiment. After 48 hours, 20 U/mL IL-2 was added to each well. Supernatants were collected after a further 24 hours for measurement by cytokine ELISA (BD Pharmingen). When selecting for T-cell clones reactive to insulin, which was used as the model antigen, Priess EBV-transformed B-cells were used (homozygous for DRB1*0401) or QBL B-cells (homozygous for DRB1*0301) in the presence of the absence of antibody (10 μg/mL anti-DR LB3.1 and anti-DQ IVD12).

Autoreactive CD4⁺ T-cells were also isolated by tetramer technology or generated in vitro using presentation of an antigen by APC. In this example of diabetes mellitus, autoreactive immune cells are insulin-specific CD4⁺ T-cells, e.g. isolated by tetramer technology (tetramer⁺), isolatable from peripheral blood. Tetramer⁺ cells can be characterized further according to their reactivity with relevant antibodies, e.g. CD45RO (memory marker), CD25 and GARP, respectively, activation or T marker.

### EXAMPLE 4

### Generation of Antigen-Specific Primed T-Helper Cells from Peripheral Lymphocytes

In accordance with Example 3, peripheral blood lymphocytes or, alternatively, lymphocytes from draining lymph nodes or spleen were isolated.

However, antigen specificity of effector T-cells was not selected for, but generated by contacting T-cells with autologous APC, that had been pulsed with the antigen. In detail, full-length peptide was be used as the model antigen, alternatively peptide fragments of the model antigen can be used, having e.g. a length of about 20 amino acids, with 10 overlapping amino acids to cover the entire length of the specific antigen by peptides that have the suitable length for presentation with HLA II. By contacting unprimed lymphocytes contained in the isolated lymphocytes, antigen-specific effector T-cells could be generated in vitro, as is known in the art.

### EXAMPLE 5

### T-Cells Having Suppressor Activity, Specifically Primed In Vitro for Suppressor Activity to Provide Immunotolerance Against a Specified Antigen

Using naïve CD4⁺ T-cells obtained according to Example 1 by sorting for CD4⁺ CD25⁻, T_{reg}-cells having a specific suppressor activity were generated, the suppressor activity of which provides for tolerance of the immune system of a recipient for that antigen.

The naïve T_{reg}-cells were primed for antigen specificity by contacting with APC which were presenting the antigen against which suppressor activity was desired. Priming with antigen-presenting APC was generally done as described in Example 4.

For induction of the suppressor phenotype, GARP was expressed subsequent to or concurrent with exposure to the APC by retroviral transduction as described in Example 7.

### EXAMPLE 6

### Expression of GARP by Viral Transduction Controls Presence of Foxp3

T_{reg}-cells, being functionally characterized by their activity for an anergic response upon TCR (T-cell receptor) stimulation and their cell-contact dependent suppressor activity, were generated from human antigen-specific Tₕₑₗₚₑᵣ-cells (denoted TₕGARP in FIG. 1) by viral transduction with a GARP-encoding nucleic acid construct. Retroviral transduction was done according to Example 7.

For comparison, Tₕₑₗₚₑᵣ-cells were retrovirally transduced with a GFP-encoding construct (denoted TₕGFP in FIG. 1) and a Foxp3-encoding construct (denoted ThFoxp3 in FIG. 2). For cell-sorting by FACS, all nucleic acid constructs included an expression cassette for GFP. After flow cytometric cell sorting, transduced cells were kept in culture and tested repeatedly for phenotypic and functional stability. For control of T_{reg}-cells, an established T_{reg}-cell-line (denoted T_{reg}THU in FIG. 2) derived from CD4+CD25high T_{reg}-cells (Ocklenburg et al., Lab. Invest. 86, 724-737 (2006)) was treated in parallel as a control.

Transduction with GARP-encoding nucleic acids results in a significant up-regulation of Foxp3 under resting conditions, comparable to Foxp3-transduced Tₕₑₗₚₑᵣ-cells and natural T_{reg}-cells, as can be seen in FIG. 1 at 10 days post stimulation with Tₕₑₗₚₑᵣ-cell-line CD4-39 for cell surface expression of CD25, and intracellular expression of CTLA4, LGALS3, and Foxp3. For cell sorting, gates were set according to isotype control antibody (CTLA4 and LGALS3) and control staining (Foxp3, clone PCH101, depicted on lower right side, thin line showing murine hybridoma T-cell transduced with GFP; thick line showing murine hybridoma T-cell transduced with human Foxp3 gene).

The cells used for analytical data of FIG. 2 were stimulated for three days with plate-bound anti-CD3 antibody and 100 U/mL IL2 and analysed for cell surface CD25 and intracellular Foxp3. Results are depicted in FIG. 3, showing up-regulation of CD25 in all three transductants, but a profound increase of Foxp3 expression only in Tₕₑₗₚₑᵣ-cells transduced with GARP or Foxp3. Accordingly, it can be inferred that over-expression of GARP, e.g. by viral transduction with a GARP-encoding expression cassette, is sufficient to induce Foxp3 and the T_{reg}-cell specific markers CD25, CTLA4 and LGALS3 in addition to GARP under resting and activated conditions.

Unlike transduction with a Foxp3-encoding expression cassette, transduction using a GARP-encoding expression cassette induced a stable regulatory phenotype in original Tₕₑₗₚₑᵣ-cells, at least over three months of in vitro antigen-specific restimtmlation and expansion. Further, it could be demonstrated that cryopreservation does not affect stability.

These results show that presence of GARP dominantly induces anergy and cell-contact dependent suppressor function in antigen-specific Tₕₑₗₚₑᵣ-cells. This finding is an essential prerequisite for medical applications of antigen-specific T_{reg} cells, e.g. engineered for a desired antigen-specificity.

The cell-contact dependent suppressor activity of T_{reg}-cells generated according to the invention was tested on GARP-transduced Tₕₑₗₚₑᵣcells (TₕGARP). Upon stimulation of TₕGARP with irradiated allogneic EBV B-cells, a severe impairment of the proliferation of TₕGARP was observed, a behaviour similar to that of Foxp3-transduced Tₕₑₗₚₑᵣ-cells. This impairment is in part reversible by presence of exogenous IL2, and it can therefore be concluded that anergy is induced by GARP. Results are shown in FIG. 4.

Proliferative impairment of TₕGARP was accompanied by the acquisition of a strong suppressor activity, equivalent to that of natural T_{reg}-cells. Results are shown in FIG. 5 and demonstrate that TₕGARP-cells impair Tₕ-cell proliferation to a similar extent as T_{reg}-cells. This suppressor function is blocked by a transwell-membrane (data not shown), which indicates that suppressor activity was cell-contact dependent. Similar results were obtained when using Tₕ-cells as responder cells instead of TₕGFP.

Further, upon down-regulation of GARP expression in human T_{reg}-cells by GARP-specific siRNA, a down-regulation of Fowp3 expression was found (data not shown). This finding confirms the dominant regulatory effect of GARP upon Foxp3.

### EXAMPLE 7

### Generating T-Cells having Suppressor Activity for a Specific Antigen from Originally Non-Regulatory T-Cells

Using originally non-regulatory T-cells obtained according to Examples 3 or 4, T-cells having an antigen specific suppressor activity could be generated to provide for immunotolerance towards that antigen. In detail, effector T-cells having antigen specificity obtainable e.g. according to Examples 3 and 4 were reprogrammed to provide for suppressor activity. For reprogramming, GARP was over-expressed in human effector T-helper cells by retroviral transduction with a coding sequence for human GARP.

For retroviral transduction, GARP was amplified from cDNA using specific primers (Seq ID No. 3) and (Seq ID No. 4) with high fidelity PFU polymerase (Promega). The PCR product was cloned into pCR4.1 TOPO (Invitrogen, Carlsbad, Calif.), sequenced and inserted into a pMSCV-based retroviral vector encoding an enhanced green fluorescent protein (eGFP) under the control of an IRES sequence. Retroviral supernatants and transfection of T-cells was performed as described previously, e.g. by Bruder et al., Eur. J. Immunol 623-630 (2004), using the amphotrophic packaging cell line PT67.

For all T_{reg}-cells, analysis of proliferation and suppressor function of T-cells transduced with GARP was performed using antigen-specific stimulation with APC with the respective Ag.

In adoption of Earle et al., Clin. Immunol. 3-9 (2005), the suppression assay used co-cultivation of a) up to 30,000 expanded T-cells manipulated according to the invention by expression of GARP or, alternatively, by reducing GARP activity by contacting with an anti-GARP antibody with b) approx. 100,000 freshly isolated PBMC serving as responder cells, plus c) approx. 100,000 APC. The APC were preferably contacted with the antigen prior to co-cultivation. APCs could be prepared from PBMC depleted of T-cells by StemSep human T-cell depletion (StemCell Technologies), followed by irradiation at 1000 rad. For a 6-7 day culture, cells were pulsed with 1 μCi ³H-thymidine.

### EXAMPLE 8

### T-Cells having No Suppressor Activity, to Provide Immunoprotection Against a Specified Antigen

In order to provide for, and preferably improve the immune response to tumor antigen, T_{reg}-cells having suppressor activity for tumor antigen were eliminated to brake the established tolerance against the tumor and enhance the anti-tumoral immune response in vaccination protocols aimed to induce tumor-specific effector CD8⁺ cytotoxic and CD4⁺ effector T-helper cells, responsive to tumor tissue for its eradication.

Here, the treatment of T_{reg}-cells contained in a pool of CD4⁺ T-cells, or isolated according to Example 2 for specificity towards the tumor antigen was done by contacting them with anti-GARP antibodies or, alternatively or additionally, with antagonists to GARP or interfering with its intracellular signalling activity to reduce the size and/or function of undesired tumor-antigen-specific Tᵢₙ- cells. Preferably, the treatment is done in vivo using a pharmceutical composition containing an antagonist to GARP expression or function, e.g. an anti-GARP antibody.

1. Use of protein comprising GARP for the production of a pharmaceutical composition.

2. Use according to claim 1, characterized in that GARP is provided by a nucleic acid encoding GARP.

3. Use according to claim 2, characterized in that the GARP encoding nucleic acid is comprised in a retroviral and/or lentiviral vector.

4. Use according to claim 1 for modifying immunoregulatory properties of CD4⁺ unprimed regulatory T-cells or non-regulatory T-cells.

5. Use according to claim 1, characterized in that the composition comprises T-cells having a suppressor activity against an antigen.

6. Use according to claim 5, characterized in that the T-cells are obtainable by a least transient expression of GARP in CD4⁺ GARP⁻-T-cells.

7. Use according to claim 4, characterized in that the T-cells are obtainable by a least transient expression of GARP in non-regulatory T-cells.

8. Use according to claim 1, characterized in that the composition is used for suppression of the immunological rejection of non-self biological material.

9. Use according to claim 8, characterized in that the non-self biological material is a transplant organ, tissue or cell.

10. Use according to claim 8, characterized in that the non-self biological material is characterized by comprising pathogenic antigen.

11. Use according to claim 1 in a process which is characterized by comprising the selection of a subset of T-cells from lymphocytes for their expression of GARP.

12. Use according to claim 10, characterized in that the subset of T-cells is GARP⁻.

13. Method for producing T_{reg}-cells having suppressor activity comprising
a. the isolation of Tₕₑₗₚₑᵣ-cells,
b. stimulation of the Tₕₑₗₚₑᵣ-cells, and
c. contacting the Tₕₑₗₚₑᵣ-cells with GARP.

14. Method according to claim 13, characterized in that the stimulation comprises contacting the Tₕₑₗₚₑᵣ-cells with APC that are provided with a pre-selected antigen.

15. Method according to claim 13, characterized by the contacting being the viral transduction with an expression cassette encoding GARP.

16. Method according to claim 13, characterized in that the Tₕₑₗₚₑᵣ-cells are obtainable by
a. the isolation of Tₕₑₗₚₑᵣ-cells from a patient sample,
b. cultivating the Tₕₑₗₚₑᵣ-cells under cell-culture conditions in the presence of the desired antigen and feeder cells, cytokines and mitogen, and
c. sorting of cultivated T_{reg}-cells to isolate CD4⁻ CFSEdim-cells.

17. Method according to claim 13, characterized in that the isolation of T_{reg}-cells is by cell sorting of GARP⁻ T-cells using an anti-GARP-antibody

18. Pharmaceutical composition, characterized by comprising T-cells having suppressor activity for a specific antigen, which T-cells are genetically manipulated with a nucleic acid construct encoding GARP.

19. Use of GARP for the production of a pharmaceutical composition for the treatment of tumor-tolerance in a patient, characterized by the composition comprising an anti-GARP antibody.
